(19) United States
(12) Patent Application Publication
(10) Pub. No.: US 2007/0190116 A1
(43) Pub. Date: Aug. 16, 2007

(54) Solid calcium bolus product with quick dissolution

(75) Inventor: John H. Lee, Olathe, KS (US)

Correspondence Address: RIGEL TECHNOLOGY CORPORATION, 10677 WIDMER, LENEXA, KS, 66215, US

(73) Assignee: Rigel Technology Corporation, Lenexa, KS (US)

(21) Appl. No.: 11/786,900

(22) Filed: Apr. 13, 2007

Related U.S. Application Data
(63) Continuation-in-part of application No. 11/263,608, filed on Nov. 1, 2005.

(51) Int. Cl. A61K 47/00; B27N 3/00

(52) U.S. Cl. 424/442; 264/109

(57) ABSTRACT

A preparation of a solid calcium bolus product with quick dissolution for preventing or treating hypocalcemia of dairy cows is described. Calcium chloride or the mixture with calcium propionate, calcium carbonate, calcium phosphate, calcium oxide, magnesium oxide, vitamin D, calcium acetate, calcium iodate, calcium lactate or their combination in particle or powder form with water at a weight ratio of 100 (dry ingredients) against 10 to 60 (water) into a liquid mass, pouring the liquid mass into moulds, setting at room temperature for 0.1 to 10 hours to form into a solid calcium product and sealing to avoid the effect from air moisture. The sealed cover is removed prior to use the solid calcium product as calcium bolus, which is dissolved into water within 0.5 hour.

### REFERENCE CITED

U. S. Patent Documents

| | | | |
|---|---|---|---|
| 4338312 | Jul. 6, 1982 | DeLuca et al. | 514/168 |
| 5395622 | Mar. 7, 1995 | Nielsen | 424/678 |
| 5560920 | Oct. 1, 1996 | Goff et al. | 424/438 |
| 6322821 | Nov. 27, 2001 | Register | 424/678 |

### OTHER REFERENCES

Block, E. 1984. Manipulating dietary anions and cations for prepartum dairy cows to reduce incidence of milk fever. J. Dairy Sci. 67:2939.

Goff, J. P. and Horst, R. L. 1993. Oral administration of calcium salts for treatment of hypocalcemia in cattle. J. Dairy Sci. 76:101.

Shaver, R. and Oetzel, G. 1990. New feed ingredients may help prevent milk fever. Hoard's Dairyman 135:344.

### CROSS-REFERENCE TO RELATED APPLICATION

The application is a continuation-in-part application of U.S. patent application Ser. No. 11/263,608, filed Nov. 1, 2005.

### BACKGROUND OF THE INVENTION

The present invention relates to the processing method of preparing a solid calcium bolus product from one or more calcium compounds with quick dissolution in water for dairy cows as calcium supplement associated with calving.

The normal calcium level in dairy cow blood is about 8 mg per 100 ml of blood. The colostrum or first milk of a fresh cow has high level of calcium and antibodies, which are very beneficial to newborn calves. In order to compensate the sudden need for calcium, it is taken from the blood stream, which reduces the calcium level in the cow blood. Low blood calcium level in the fresh cows may cause hypocalcemia, which is commonly called milk fever. It is associated with the sudden onset of lactation and usually occurs within 72 hours after calving. If treatment for such cases is not immediate and successful, there may be a danger to the cows. Treatment in all cases attempts to bring the blood calcium level to normal.

It is well known to treat hypocalcemia or milk fever by intravenous dosing (IV) such as calcium gluconate solution, which is generally administered by a veterinarian. It takes time due to the slow administration rate. The calcium from the treatment is released quickly and immediately after the IV. The solution (23% calcium gluconate) contains low calcium level such as 2.1% (23%×40/430). 250 ml of calcium gluconate solution contains only 5.3 grams of calcium. It tends to spike a large amount of calcium to the blood. The cows may respond to the spike by releasing a hormone (calcitonin), which temporarily prevents the cows from naturally restoring their own blood calcium levels. So the relapse may occur again after the IV treatment. The IV is a temporary solution and not good for hypocalcemia prevention.

Another treatment is by oral administrating aqueous calcium chloride solution, gel or solid bolus products to the cows. Then the cows can replace the calcium loss in their blood by intestinal calcium absorption.

Aqueous calcium chloride solution has a large volume and bitter taste and is unpleasant. Therefore the cows must be force-treated. Viscous calcium gels are forced from a caulking or drench gun and placed over the cow's tongue. Timing may cause the cows to reject. Partial products may be spit out and wasted by the cows. If the calcium liquid enters the lungs, it may result in aspiration pneumonia and death.

Solid calcium boluses are orally administrated by balling guns, which takes much short time. Solid calcium boluses contain more calcium than calcium solution or gel products. There are two commercial solid calcium bolus products in the current U.S. market. One solid calcium bolus product is made from calcium chloride and calcium sulfate with an encapsulated coating such as non-glyceride ester of vegetable fatty acids or glyceryl PEG ricinoleate, which is produced by a Denmark company under U.S. Pat. No. 5,395,622. Each commercial bolus (190 grams) contains 43 grams of calcium. The calcium bolus product is dissolved in water very slow, which begins to release after 30 minutes and to be dissolved into water after 400 minutes. Another solid calcium bolus product is made from calcium chloride with a paper board as holding and packaging material, which is produced by another Denmark company under a patent pending. Each commercial bolus (166 grams) contains 40 grams of calcium. The calcium bolus product is dissolved in water extremely slow, which begins to release after 400 minutes and to be dissolved after overnight. The encapsulated coating and paper board materials reduce the chemicals to be contacted with water and dissolved into water significantly. New solid calcium bolus products are needed to be developed for more convenience and quick releasing calcium.

A number of patents have been issued for some attempts. U.S. Pat. No. 6,322,821 discloses a method for a calcium liquid product containing calcium chloride, propylene glycol, B vitamins and minerals, which is administered using a drench gun. U.S. Pat. No. 5,560,920 discloses a method for a calcium liquid drench containing calcium propionate, propylene glycol, citric or phosphoric acid, and sodium chloride for treating hypocalcemic disorder associated with the onset of lactation in dairy cows. U.S. Pat. No. 5,395,622 discloses a preparation of calcium chloride (binding with water) and calcium sulfate (binding with water) mixed with water into a pumpable mixture, introduced into moulds and heated the molds so that the mixture solidifies into a solid calcium bolus product, which is then respectively encapsulated within individually gelatine capsules. The product is used for treatment or prevention of hypocalcemia in ruminants. The encapsulating material provides a smooth surface and limits the moisture effect from air. But the encapsulating material also causes two major problems. One is the melting problem at a temperature of 25 degree C. or above. Another problem is the layer of the encapsulating material causes a barrier for slow releasing calcium. Water have to pass through the encapsulating material to contact and to dissolve calcium chloride and calcium sulfate, which takes long time. U.S. Pat. No. 4,338,312 discloses a method for prophylactically treating dairy cattle for the milk fever by administrating to the cattle a 25-hydroxylated vitamin D compound and a 1.alpha.-hydroxylated vitamin D compound in combination in an amount sufficient to induce said prophylaxsis.

The calcium density in solid calcium bolus products is significantly high compared with the calcium solution or gel products. A solid calcium bolus in such as a cylinder, which could be 14 cm long and 3 cm in diameter and contain 40-50 grams of calcium, may be orally administrated to the cows by a balling gun. It takes much less time to administrate the solid calcium bolus products orally to the cows compared with the IV and administrating aqueous calcium chloride solution or gel products. Another issue is how to release calcium as quick as possible for the cows to absorb later.

### SUMMARY OF THE INVENTION

It has now been found that a solid calcium chloride product itself or with nutritional ingredients of calcium carbonate, calcium phosphate, calcium propionate, calcium oxide, vitamin D, magnesium oxide, calcium acetate, calcium iodate, calcium lactate or their combination may be produced from an aqueous mixture. The present invention overcomes the problems of the current calcium bolus products and provides a novel process to convert the calcium compound(s) in particle or powder form into a solid calcium product as a calcium bolus product. Also this process in this invention makes the calcium bolus product to be dissolved into water or liquid quickly.

Calcium chloride with or without binding water in particle or powder form is mixed with water at 10 to 60 (water) against 100 (calcium chloride) range on a weight basis into a liquid mass and is poured into moulds. The moulds may have test tube shape with such as a 1-3 cm diameter, 5-15 cm length, rounded bottom and empty hole. The moulds may have different shapes and sizes. The liquid mass forms a hard solid as a solid calcium bolus product after setting at room temperature for a period of time from 0.1 to 10 hours. In the most cases, the solidification from the liquid mixture mass into a solid form needs about ½ to 3 hours.

Calcium chloride with or without binding water is mixed with other calcium ingredients of calcium phosphate, calcium propionate, calcium carbonate, calcium oxide, calcium acetate, calcium iodate, calcium lactate or their combination in particle or powder form at 0.01 to 300 (other calcium ingredients) against 100 (calcium chloride) on a weight basis to form a mixture. Then above calcium mixture is mixed with water at 10 to 60 (water) against 100 (all dry ingredients) on a weight basis to form a liquid mass and is poured into moulds. The liquid mass forms a hard solid as a solid calcium bolus product after setting at room temperature for a period of time such as from 0.1 to 10 hours. In the most cases, the solidification from the mixture mass in a sludge form into a solid form needs about ½ to 3 hours.

Mould material can be plastic, paper, wood or metal. The molds made from thin plastic or aluminum foil paper can be sealed by a sealer after forming the solid calcium bolus product in the molds to avoid the moisture effect from air. The process with the molds of thin plastic or aluminum foil paper with smooth inside surface is more convenient to be sealed directly at low cost for less labor and package material. The moisture affects the solid calcium product to cause swell, crack or wet situations on the product surface. The moisture effect should be eliminated or reduced. When the molds can not be sealed directly, the solid calcium bolus product is needed to be removed from the molds and an additional packing process with bags are needed. The bags can be made from plastic or aluminum foil paper with sealing function. Vacuum package is better method to be used to avoid the moisture to affect the solid calcium bolus product. A coating process with vegetable oil onto the solid calcium bolus product avoids or reduces the moisture effect for short term.

In mould materials, plastic provides better sealing, handling, timing, and flexibility for this application. It has been found the calcium bolus product with the sealed plastic mould is stable without any physical change at room temperature even after 550 days. After removing the plastic mould, the solid calcium bolus product begins to release into water almost immediately after contacting with water. The solid calcium boluses are usually dissolved into water after 15 minutes to have a quick dissolution, which is much quick than other two commercial solid calcium bolus products. One of the commercial calcium bolus product begins to release after 30 minutes and to be dissolved after 400 minutes. Another commercial solid calcium bolus product the paper board begins to release after 400 minutes and to be dissolved after overnight.

Calcium chloride and calcium propionate are water soluble, which release quick in water or the cows. Calcium phosphate and calcium carbonate are not water soluble, which release slow in water or the cows. After the solid calcium bolus product is released into water, water soluble calcium ingredients go into water and non-water soluble calcium ingredients in very fine form suspend into water. Quick and slow release calcium ingredients are good combination for the cows to absorb calcium over the time. An additional ingredient may be also added into the product. It is well known that phosphate, vitamin D and magnesium also contribute to treat or prevent hypocalcemia (milk fever) of the cows besides the major factor calcium.

### DETAIL DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

Calcium chloride (800 grams) was mixed with water (176 grams) into a liquid mass. The ratio of water against calcium chloride was 22 to 100 (176/800). The liquid mass was poured into plastic moulds with internal diameter of 3.2 cm. After setting at room temperature for two hours, solid calcium product was formed. The plastic moulds were sealed by a heat sealer.

### EXAMPLE 2

Calcium chloride (700 grams) was mixed with calcium carbonate (300 grams). The ratio of calcium carbonate against calcium chloride was 43 to 100 (300/700). Then water (210 grams) was mixed with the above mixture of calcium chloride and calcium carbonate into a liquid mass. The ratio of water against the dry ingredients of calcium chloride and calcium carbonate was 21 to 100 (210/1000). The liquid mass was poured into plastic moulds with internal diameter of 2.5 cm. After setting at room temperature for one and half hours, hard solid calcium product was formed. Then the plastic moulds were removed. The solid calcium boluses were coated with soybean oil, packed into plastic bags and sealed by vacuum sealer. The solid calcium bolus product was set in room for 550 days without any physical changes.

### EXAMPLE 3

Calcium chloride (570 grams) was mixed with calcium carbonate (200 grams) and calcium propionate (30 grams). Then water (160 grams) was mixed with the above mixture of calcium chloride, calcium carbonate and calcium propionate into a liquid mass. The ratio of calcium carbonate and calcium propionate against calcium chloride was 40 to 100 ((200+30)/570). The ratio of water against the dry ingredients of the calcium mixture was 20 to 100 (160/(570+200+30)). The liquid mass was poured into the plastic moulds (tube form) with internal diameter of 3.2 cm and rounded bottom. After setting at room temperature for two hours, a solid calcium product was formed. The plastic moulds were sealed by a heat sealer. After two days, the plastic mould was removed. The solid calcium bolus product with rounded bottom (180 g at 3.2 cm diameter and 14 cm length) was placed into 1200 ml water. The bolus begun to release into the water after about 5 seconds. The bolus disappeared into the water after about 15 minutes.

What is claimed is:

1. A method of preparing a solid calcium bolus product for administering to dairy cows, said method comprising the steps of
(a) mixing calcium chloride with the ingredients of calcium propionate, calcium carbonate, calcium phosphate, calcium oxide, magnesium oxide, vitamin D, calcium acetate, calcium iodate, calcium lactate or their combination in particle or powder form at 100 (calcium chloride) against 0 to 300 (said ingredients) on a weight basis to form a mixture;
(b) adding water to said mixture at 10 to 60 (water) against 100 (said mixture) on a weight basis and mixing into a liquid mass;
(c) pouring said mass into moulds; and
(d) setting said mass in said moulds for 0.1 to 10 hours to form the solid calcium bolus product.

2. The method of claim 1 wherein the moulds are made from plastic, paper, wood or metal.

3. The method of claim 1 wherein the solid calcium bolus product is coated with vegetable oil.

4. The method of claim 1 wherein an ingredient is added.

5. The method of claim 1 wherein a heat, pressure or vacuum seal is used to avoid the moisture effect from air to the solid calcium bolus product.

6. A method of preparing a solid calcium bolus product comprising sealing a package to separate the solid calcium bolus product from the air moisture effect and dissolving the solid calcium bolus product into water to have its dissolution within two hours after removing the package and contacting into the water.

7. The method of claim 6 wherein the package is made from plastic or aluminum foil.

8. The method of claim 6 wherein a heat, pressure or vacuum seal is used for the package to avoid the solid calcium bolus product to have direct contact with the moisture from air.

9. The method of claim 6 wherein the solid calcium bolus product has direct contact without a barrier when placing the solid calcium bolus product into water to have its dissolution into the water within two hours after removing the package.
